(19) United States
(12) Patent Application Publication
(10) Pub. No.: US 2009/0293139 A1
(43) Pub. Date: Nov. 26, 2009

(54) DOUBLE-INDUCIBLE GENE ACTIVATION SYSTEM AND ITS APPLICATIONS

(75) Inventors: Jiang Chang, Houston, TX (US); Robert J. Schwartz, Houston, TX (US); Viraj R. Shah, Houston, TX (US)

Correspondence Address: JACKSON WALKER LLP, 901 MAIN STREET, SUITE 6000, DALLAS, TX, 75202-3797, US

(73) Assignee: The Texas A&M University System Agency State of Texas, College Station, TX (US)

(21) Appl. No.: 12/418,403

(22) Filed: Apr. 3, 2009

Related U.S. Application Data
(60) Provisional application No. 61/123,047, filed on Apr. 3, 2008.

(51) Int. Cl. C12N 15/00; C12N 15/63

(52) U.S. Cl. 800/22; 435/320.1

(57) ABSTRACT

A double-inducible system for expressing a transgene, preferably comprising an RU486-inducible system integrated with a CID-inducible system. The invention further comprises a gene expression system for use in in vitro cell culture studies, and a gene expression expression system for use in engineering modified bigenic mice.

The present invention claims priority to U.S. Provisional Patent Application Ser. No. 61/123,047, filed Apr. 3, 2008, the entire content of which is hereby incorporated by reference.

### STATEMENT OF RIGHTS TO INVENTIONS MADE UNDER FEDERALLY SPONSORED RESEARCH

The present invention used, in parts, funds from the National Institutes of Health (contract grant numbers PO1-HL49953, RO1-HL64356, to RJS) and an American Heart Association Scientist Development Grant (contract grant number 0335155N, to JC). The government may have certain rights in the invention.

### FIELD OF THE INVENTION

The present invention relates generally to the fields of molecular biology and genetic engineering, and more specifically to double-inducible gene expression systems.

### BACKGROUND

A major goal of genetic engineering of animals and cultured cells is manipulation or replacement of gene expression. Towards that goal, exquisite, non-leaky temporal and spatial control of gene expression could minimize artifacts and increase utility by eliminating basal signaling and potential toxicities.

Several strategies have been utilized to achieve this goal. Previous studies have demonstrated the ability of the mifepristone (RU486)-inducible system to control spatial and temporal transgene expression. This system uses a chimeric transcription factor that can reversibly bind to a target gene promoter to allow for regulation of transgene expression upon administration of RU486 (FIG. 1). This type of on and off regulation can be achieved in any cells or tissues along with the use of a tissue-specific promoter.

Another inducible system includes a chimeric precursor, such as caspase 3, harboring a dimer binding domain, and a chemical inducer of dimerization (CID) that can bind to the dimmer binding domain (or CID binding domain (CBD)) to bring two molecules of caspase 3 together to form a dimer, and subsequently initiate caspase-3 self-activation through internal proteolysis (FIG. 1b). The precursor will remain biologically inactive until its exposure to CID.

Both of these inducible systems are specific, reversible, non-toxic, and have a relative induction potential. However, an important drawback of each system is the potential for leakage of transgene expression, which is the major obstacle for the application of many inducible gene induction technologies.

The present invention relates to a double-inducible gene activation system and a transgenic mouse line harboring such a double-inducible gene activation system. While the double-inducible activation system retains all of the advantages of both inducible systems, it compensates for each system's drawbacks, resulting in highly inducible, efficient, and stringent gene expression.

In an embodiment of the present invention, temporal and spatial control of a gene on/off at transcriptional level and translational level are integrated into one system, which has been demonstrated to be unexpectedly more stringent and efficient than the current individually inducible systems.

In a preferred embodiment of the invention, a double-inducible gene activation system controls a gene on and off at two levels: the transcriptional level and the posttranslational level. In the gene transcriptional level, the target gene will be only transcribed upon the addition of the first inducer, RU486. However, this final protein will not be activated (inactive form or precursor) until the addition of the second inducer, CID, which modifies the precursor by dimerization. By controlling a gene transcription and a posttranslational modification, we can achieve a highly tight, leakage-free gene expression control.

### SUMMARY

The present invention comprises a system for double-inducible gene activation, preferably achieved through the integration of a transactivation-based inducible system and a dimerization-based inducible system. The invention further comprises a gene expression system for use in in vitro cell culture studies, and a gene expression expression system for use in engineering modified bigenic mice.

In a preferred embodiment, the invention comprises a double-inducible gene activation system containing an RU486-inducible system and a chemical inducer of dimerization (CID)-inducible system. Through these dual systems, the invention comprises a double barrier to target gene expression, which may be beneficial in preventing leakage of target gene expression under conditions which are not meant to promote target gene expression.

In a further preferred embodiment, the invention may comprise a bigenic mouse. The bigenic mouse may be the product of a cross between a first and a second transgenic mouse. The bigenic mouse may express the target transgene, regulated by both the first and the second inducible system. In a most preferred embodiment, the bigenic mouse may be either a K14-Glp65/iCasp3 or K14-Glp65/iCasp9 bigenic mouse.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a preferred embodiment of the invention including the chimeric transcription factor Glp65 with the ligand binding domain of the truncated human progesterone receptor (PR-LBDΔ), the GAL4 DNA binding domain, and the Glp65 transactivation domain of NF-κB is placed under the control of a keratin 14 (K14) promoter. CBD: CID binding domain;

FIG. 2 shows a diagram of a preferred embodiment of the invention showing that the addition of chemical inducer of dimerization (CID) forces aggregation of the chimeric caspase precursors, initiating self-activation;

FIG. 3 shows representative Western blots showing the target caspase-9 gene induction and activation in a preferred embodiment of the invention;

FIG. 4 shows representative Western blots showing the target caspase-3 gene induction and activation in a preferred embodiment of the invention;

FIG. 5 shows caspase-3 activity under three different induction protocols in a preferred embodiment of the invention. *P<0.05 compared to control;

FIG. 6 shows caspase-9 activity under three different induction protocols in a preferred embodiment of the invention. *P<0.05 compared to control;

FIG. 7 shows induction of transgenic caspase-3 in mouse skin tissues compared to their endogenous ones in a preferred embodiment of the invention;

FIG. 8 shows induction of transgenic caspase-9 in mouse skin tissues compared to their endogenous ones in a preferred embodiment of the invention;

FIG. 9 shows microscopic and histological analysis of skin from adult mouse ear.Skin sections from K14-Glp65/iCasp3 adult mouse ears were visualized by H&E stain (panels a-c), and were immunostained by keratin 14 antibody (panel d-f). Caspase-3 induction (panel g) and activation (panel i) were shown by immunohistochemical staining with anti-HA antibody. HA-positive dark brown cells were indicated by arrows. The skin apoptosis was evaluated by TUNEL assay (panels j-l). Activated caspase 3 was detected by a specific antibody exclusively against active form of caspase 3 (red indicated by arrows) (panels m-o); and

FIG. 10 shows microscopic and histological analysis of skin from newborn back skin in a preferred embodiment of the invention. Skin sections from K14-Glp65/iCasp9 newborn mice back skin were visualized by H&E stain (panels a-c), and were immunostained by keratin 14 antibody (panels d-f). Caspase-9 induction (panel g) and activation (panel i) were shown by immunohistochemical staining with anti-HA antibody. HA-positive dark brown cells were indicated by arrows. The skin apoptosis was evaluated by TUNEL assay (panels j-l). Activated caspase 9 was detected by a specific antibody exclusively against active form of caspase 9 (red indicated by arrows) (panels m-o).

### DETAILED DESCRIPTION OF PREFERRED EMBODIMENTS

The present invention comprises a system for double-inducible gene activation, preferably achieved through the integration of a transactivation-based inducible system and a dimerization-based inducible system. The invention further comprises a gene expression system for use in in vitro cell culture studies, and a gene expression expression system for use in engineering modified bigenic mice.

In a preferred embodiment of the invention, a double-inducible gene activation system controls a gene on and off at two levels: the transcriptional level and the posttranslational level. In the gene transcriptional level, the target gene will be only transcripted upon the addition of the first inducer, RU486. However, this final protein will not be activated (inactive form or precursor) until the addition of the second inducer, CID, which modifies the precursor by dimerization. By controlling a gene transcription and a posttranslational modification, we can achieve a highly tight, leakage-free gene expression control.

In an embodiment of the present invention, temporal and spatial control of a gene on/off at transcriptional level and translational level are integrated into one system, which has been demonstrated to be unexpectedly more stringent and efficient than the current individually inducible systems.

In a preferred embodiment, the invention comprises a double-inducible gene activation system containing an RU486-inducible system and a chemical inducer of dimerization (CID)-inducible system. Through these dual systems, the invention comprises a double barrier to target gene expression, which may be beneficial in preventing leakage of target gene expression under conditions which are not meant to promote target gene expression.

In a highly preferred embodiment, the invention comprises a first inducible system, which includes an RU486-inducible system comprising a progesterone receptor ligand binding domain (PR-LBD) in communication with a Glp65 transactivation domain. In this embodiment, the first inducible system is capable of activating a Gal4 domain which regulates a transgene in the presence of RU486. This embodiment further comprises a second inducible system, which includes the transgene, comprising a precursor protein and dimerization domain capable of dimerizing and self-activating in the presence of a chemical inducer of dimerization (CID).

The transgene in this embodiment may be a caspase precursor, most preferably a precursor of caspase-3 in communication with a CID binding domain (CBD) or a precursor of caspase-9 in communication with a CBD. The caspase precursor and CBD are expressed as a fusion protein. The caspase precursor in communication with the CBD is expressed in response to induction of the first inducible system by RU486, and the caspase precursor is capable of dimerizing and self-activating in the presence of CID through the CBD.

In a further preferred embodiment, the invention may comprise a bigenic mouse. The bigenic mouse may be the product of a cross between a first and a second transgenic mouse.

The first transgenic mouse may express a tissue-specific chimeric transcription factor comprising Glp65, wherein the transcription factor can be activated by RU486 through a PR-LBD. In a preferred embodiment, the chimeric transcription factor is driven by the epidermal-specific kertin 14 (K14) promoter.

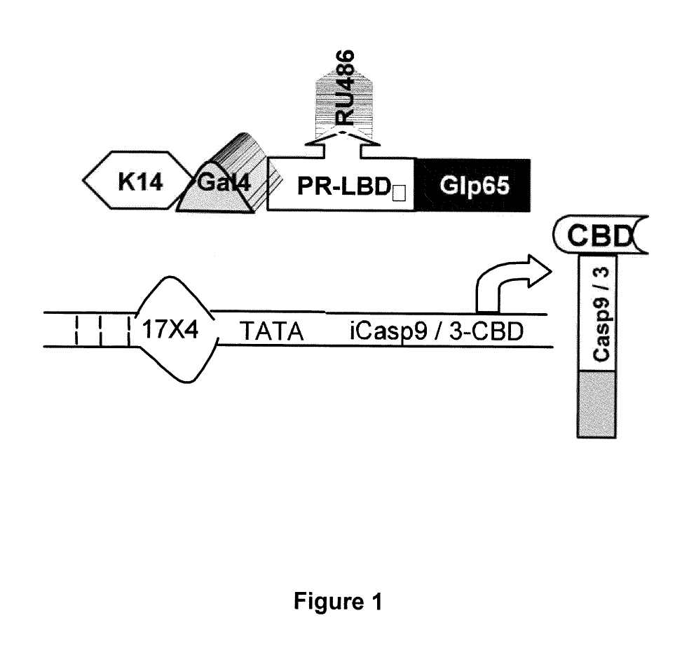

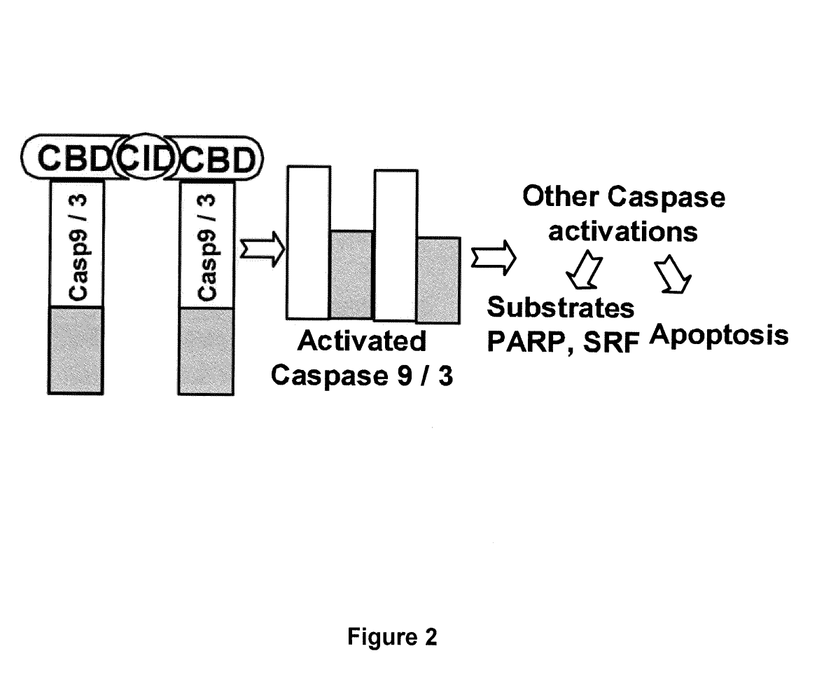

The second transgenic mouse may carry a target transgene which comprises a precursor protein capable of dimerization and self-activation in the presence of a CID, In a preferred embodiment, the transgene consists of either inducible caspase-3 or caspase-9 precursors in communication with a CBD (FIGS. 1 and 2).

In a most preferred embodiment, the bigenic mouse may be either a K14-Glp65/iCasp3 or K14-Glp65/iCasp9 bigenic mouse. The bigenic mouse may express the target transgene, regulated by both the first and the second inducible system.

### DEFINITIONS

K14, or keratin 14 promoter as used herein describes a promoter derived from the promoter of the keratin 14 gene, or an epidermal-specific promoter, or any other promoter capable of driving gene expression in epidermal cells.

Gal4, or Gal4 transcription factor as used herein describes a transcription factor with similar activity to, or which is derived from yeast.

Gal4 DNA binding domain as used herein describes a specific DNA region that can be exclusively bound by Gal4 transcription factor.

PR-LBDΔ as used herein describes the ligand binding domain of a truncated human progesterone receptor, or a domain that is derived from, or binds RU-486 in a similar manner to a human progesertone receptor, or a domain that binds RU-486 at a rate sufficient to result in activation of a fused transactivation factor.

The term Glp65 as used herein describes a transactivation domain of transcription factor NF-kB.

The term HA as used herein describes the influenza protein hemaglutinin, a protein epitope tag.

The term chemical inducer of dimerization (CID), as used herein describes a lipid-permeable dimeric ligand.

The term CID-binding domain (CBD), as used herein describes a specific motif or a domain of a protein that is allowed CID binding.

The term DVPD, as used herein, refers to a caspase cleavage site. D stands for aspartic acid, V stands for valine and P stands for proline.

### Example 1

### Preferred Embodiment of the Invention In Vitro

A double-inducible gene activation system was evaluated in vitro using a cell culture model. Three plasmids were constructed: 1) chimeric transcription factor, Glp65 containing progesterone receptor ligand-binding domain (PR-LBDΔ) (FIG. 1); 2) pTATA-HA-iCasp3 (myristoylated) carrying CBD; and 3) pTATA-HA-iCasp9 carrying CBD (FIGS. 1 and 2). The chimeric transcription factor and one of the two caspase plasmids were transfected into CVI cells (monkey kidney fibroblasts).

One plasmid expressed a tissue-specific chimeric transcription factor (Glp65) that can be activated by RU486, which functioned as the first induction in our system. In this case, an epidermal-specific keratin 14 (K14) promoter was used to direct gene expression to the keratinocytes of the basal epidermis and hair follicles (KI4-Glp65, FIG. 1; Cao et al., 2002; Kucra et al., 1996). The second plasmid expressed the target transgene. The plasmid contained four copies of the 17-mer GAL4 binding site in the promoter region (FIG. 1). Full length of caspase-3 cDNA with CID binding domain and HA-tag fragment was cloned by polymerase chain reation (PCR) from an expression vector pSH1/M-Fv2-Yama-E (Fan et al., 1999) and was then subcloned into p17×4 TATA-H2 kd vector (Bo et al., 2005) by ClaI and BamH I to generate pTATA-HA-iCasp3 mice.

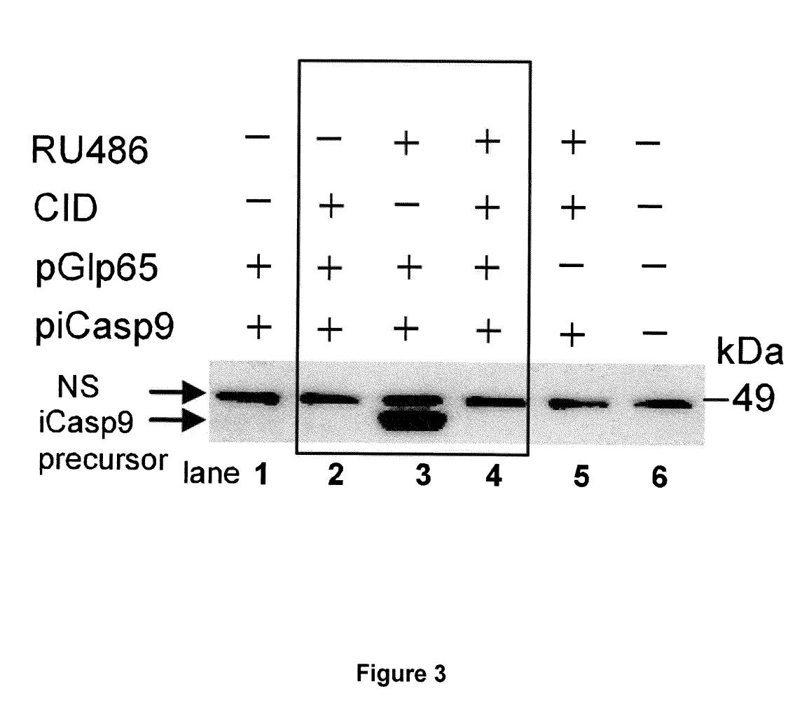

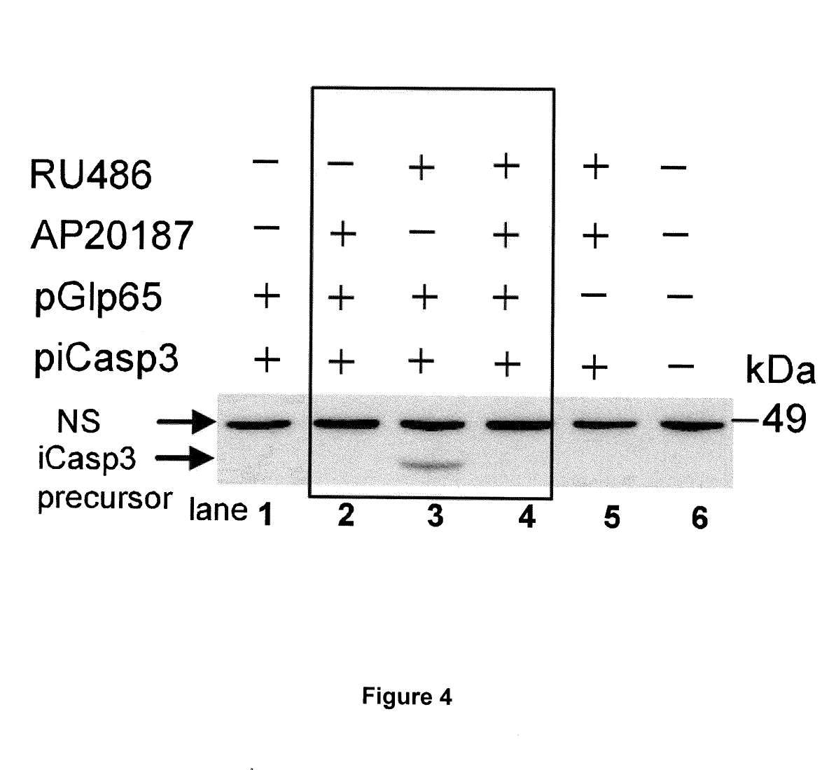

Cell lysates were assessed by Western blot using an anti-HA antibody. As shown in FIGS. 3 and 4, RU486 induced the expression of iCasp9 (FIG. 3, lane 3) or iCasp3 precursors (FIG. 4, lane 3). These precursors remain inactive without the addition of CID. With the subsequent application of CID, caspase 3 and caspase 9 were activated as evidenced by the disappearance of the caspase precursors due to the hemagglutinin (HA) tag carrying a caspase cleavage consensus site, DVPD (lanes 4 in FIGS. 3 and 4). No caspase precursors were induced with the application of CID alone (lanes 2 in FIGS. 3 and 4).

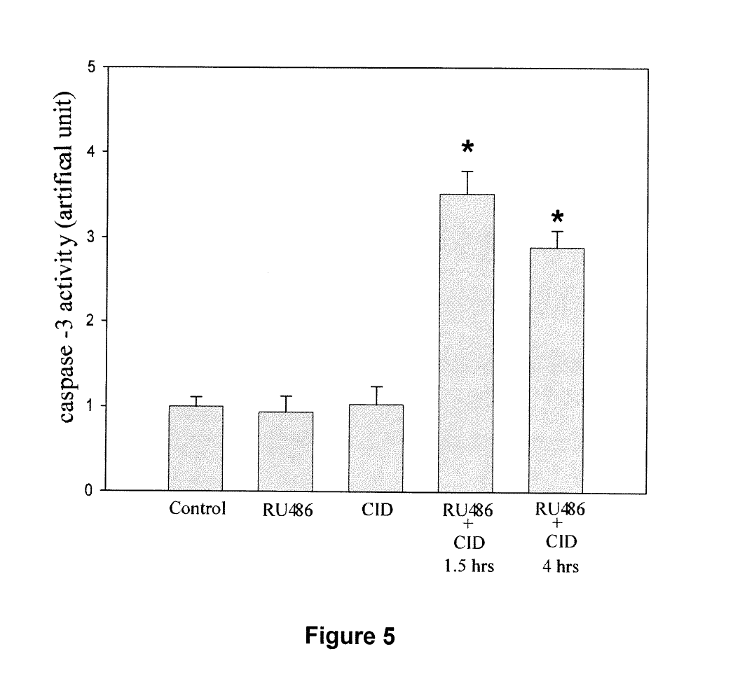

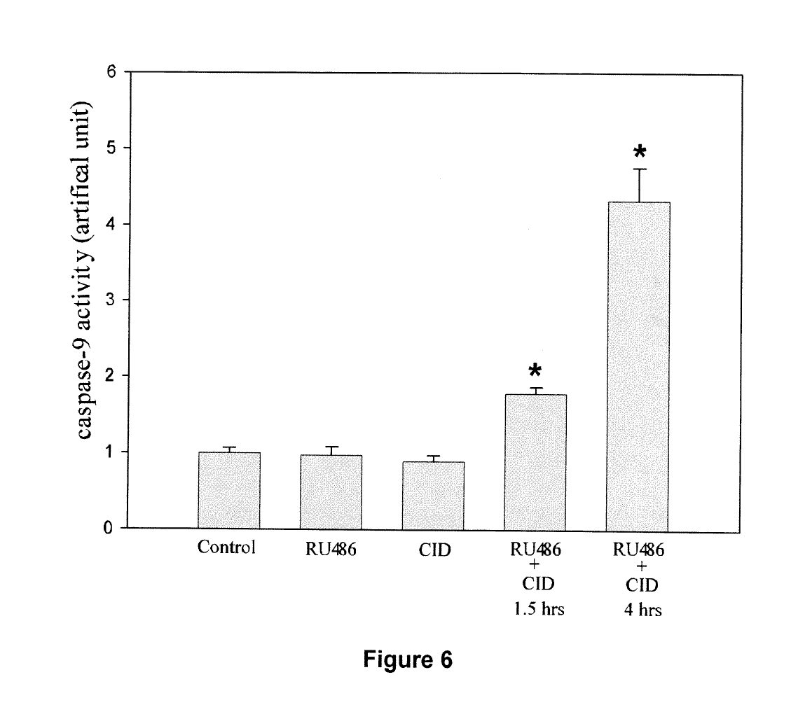

Because of this caspase cleavage consensus site within the HA tag, activation of caspase 3 and caspase 9 were further verified by using Clonetech ApoAlert Caspase Assay as shown in FIGS. 5 and 6. Three-fold and two-fold increases in caspase-3 and capase-9 activity were observed in cells treated with both drugs for 1.5 hrs, but not in cells treated with either RU486 or CID alone. A continued increase in caspase-9 activity was detected after 4 hrs of dual-drug treatment (FIG. 6) compared to a slight decrease in caspase-3 activity suggesting a different activation pattern between the two caspases.

### Example 2

### I Preferred Embodiment of the Invention In Vivo

A double-inducible gene activation system was tested in vivo in two bigenic mouse lines, K14-Glp65/iCasp3 and K14-Glp65/iCasp9. The bigenic mice were generated by breeding two individual mouse lines.

The first transgenic mouse line expressed a tissue-specific chimeric transcription factor comprising Glp65, which can be activated by RU486 through a PR-LBD. In this embodiment, the chimeric transcription factor is driven by the epidermal-specific kertin 14 (Ki4) promoter, which directed gene expression to the keratinocytes of the basal epidermis and hair follicles (K14-Glp-65)(FIG. 1).

The second transgenic mouse line carried a transgene. For the second iduction, caspase-3 and caspase-9 mice were generated containing four copies of the 17-mer GAL4 binding site in the promoter region (FIG. 1). Full length of caspase-3 cDNA with the CID binding domain anHA-tag fragment was cloned by PCR from an expression vector pSH1/M-Fv2-Yama-E (Fan L., Hum Gene Ther. 1999, 10:2273-85) and was then subcloned into p17×4 TATA-H2 kd vector (Bo J., J Mol Cell Cardiol 2005, 38:685-91) by ClaI and BamH I to generate pTATA-HA-iCasp3 mice. The final DNA fragment for microinjection was cut out by SphI and EcoRI. The same strategy was applied for the generation of the inducible caspase-9 construct.

The breeding of the first and second transgenic mouse lines generated bigenic mice called K14-Glp65/iCasp3 and K14-Glp65/iCasp9, which harbored both RU486 and CID regulators as depicted in FIG. 1. With the application of RU486, the Glp65 fusion protein was exclusively expressed in skin keratinocytes, which subsequently initiated caspase precursor induction. In the presence of lipid-permeable dimeric ligand CID (AP20187, ARIAD Pharmaceuticals), which was applied intraperitoneally to adult mice or daubed topically to the back skin of neonates, the inactive caspase was forced to dimerize, leading to an autoproteolysis and self-activation.

Bigenic mice between 8 and 15 weeks of age were subjected to RU486 treatment using a 21-day-release pellet that was surgically inserted beneath the skin in the region of the posterior scapula. The addition of RU486 activates the chimeric transcription factor and allows it to bind to the GAL4 DNA binding site of the caspase precursor target gene (either icasp3 or icasp9) and induces target gene expression. After seven days of RU486 treatment, CID was applied intraperitoneally to induce self-activation of caspase precursors. Placebo pellets lacking RU486 and CID-diluent were used for control groups. Five days following application of CID, reddening of the surface layers of the skin and thickness was observed on the skin covering the ears. We did not observe this phenotype in either control mice or in bigenic mice that only received RU486 treatment.

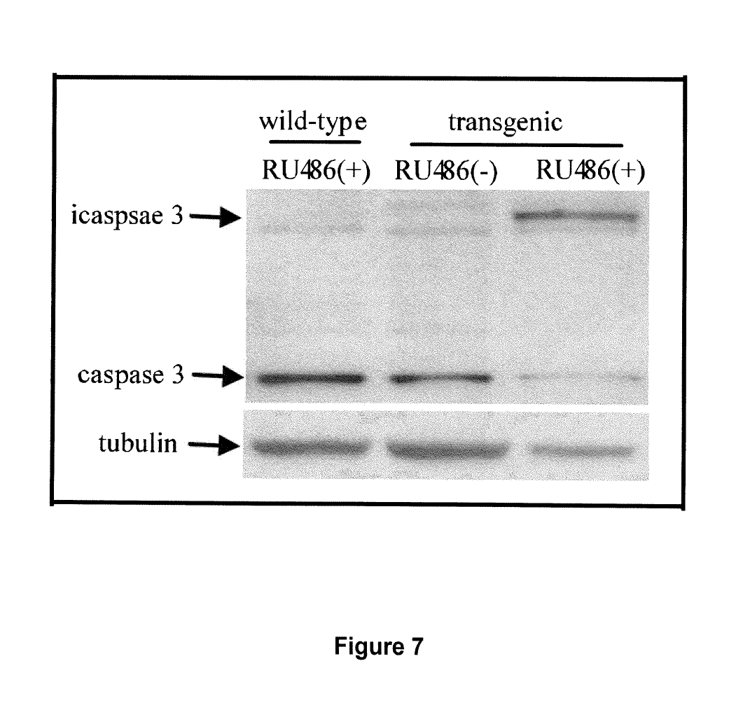

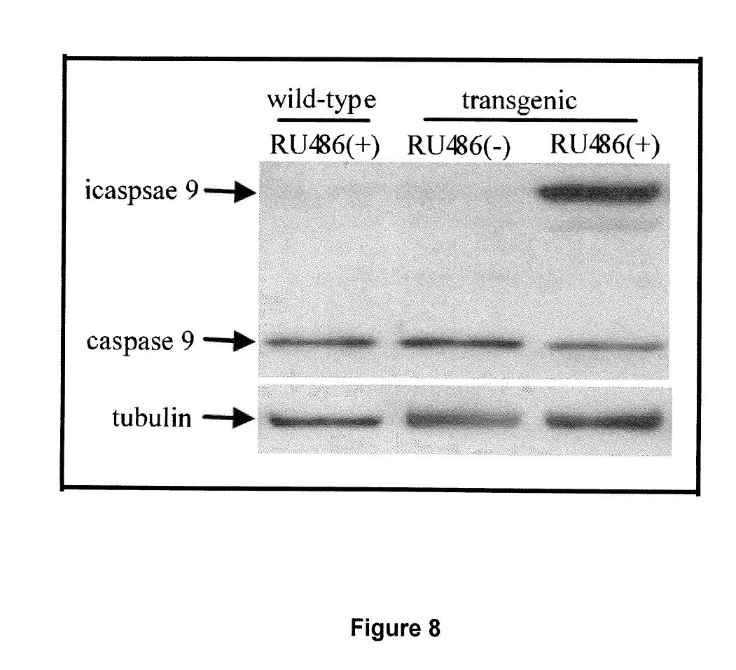

To closely monitor the phenotypic changes of the skin of bigenic mice after the activation of caspases through the double-inducible system, we tested system in neonates by delivering RU486 in utero. The RU486 was dissolved in sesame oil and delivered intraperitoneally for 5 days at a dosage of 100 μg/kg per day into pregnant female K14-Glp65/iCasp3 and K14-Glp65/iCasp9 bigenic mice at 14.5 days gestation. To counter the abortion side-effect of RU486, progesterone (Sigma, St. Louis, Mo.) at 0.5 mg/mouse per day was applied along with RU486. Transgenic pups were treated topically with CID on their dorsal anterior-posterior (AP) axes twice per day and monitored closely for phenotypic changes or visible signs of apoptosis. Control littermates were treated with only the diluent only without CID. After two days of CID induction, the skin of the CID-treated pups exhibited peeling and appeared dehydrated when compared to the skin of the control littermates. On day four, skin biopsies were taken from the back skin of the pups and fixed in 4% paraformaldehyde. The expression levels of induced caspase 3 or caspase 9 were evaluated by Western blot as shown in FIGS. 7 and 8, strong expression of both conditional proteins were detected exclusively in RU486-treated transgenic mice, but not in wild-type mice with RU486 or transgenic mice without RU486 treatment.

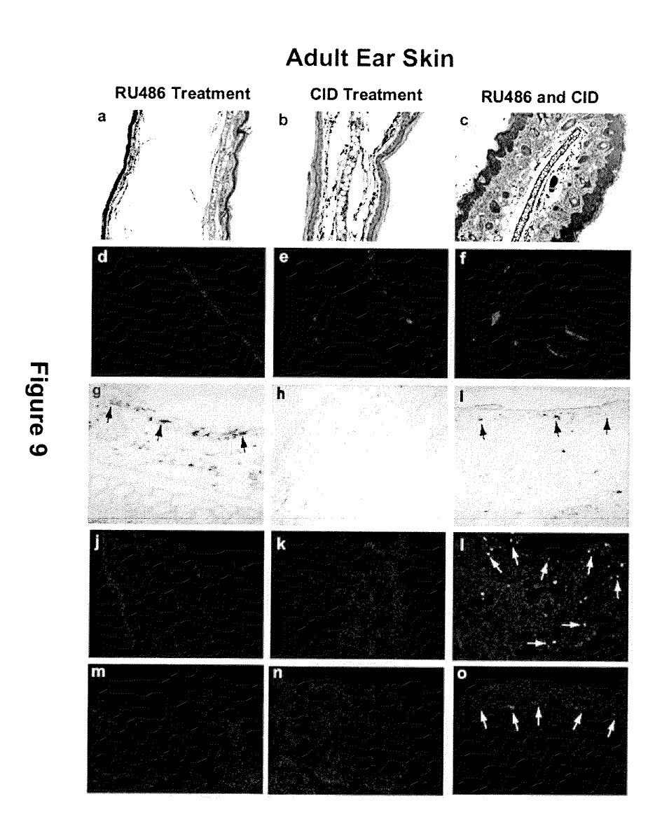

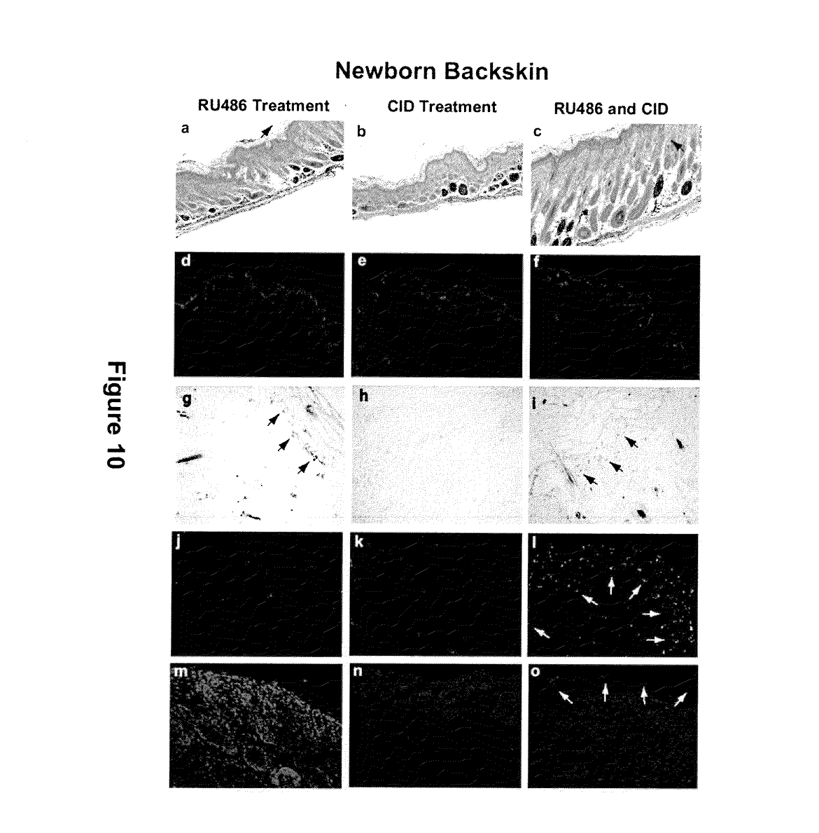

Induction of the two caspases was then further studied by immunohistochemical staining with an anti-HA antibody. The induced activation of caspases was probed using two antibodies exclusively against active forms of caspase 3 and caspase 9, respectively. Apoptosis was evaluated by TUNEL staining. The results with three different induction protocols from adult mouse ear skins and newborn mouse back skins were summarized as follows and in FIG. 9 and FIG. 10:
Strong expression of caspase precursors was observed in RU486-alone treated group (FIGS. 9g and 10g) but not in CID-only treated group.
Less straining was found in dual-drug treated groups indicating caspase cleavage of the HA tag (FIGS. 9h and 10h). After treatment with both RU486 and CID, the skin became hyperplasic (FIGS. 9c and 10c).
Significant increases in TUNEL-positive nuclei (green) were observed in mice with the dual-drug treatment (FIGS. 21 and 31), but not in either the RU486-only (FIGS. 9j and 10j) or the CID-only (FIGS. 9k and 10k) treated group, which displayed normal skin structures with no or basal level of apoptosis.
Consistent with TUNEL assay, great increases in active caspase 3 and caspase 9 were detected using antibodies specific for caspase-3 and caspase-9 active forms in dual-drug treated skins (FIGS. 9o and 10o).
The apoptotic cells were predominantly localized at epidermal layers that corresponded with K14 expression in basal cells (FIGS. 9d-9f and 10d-10f).

These results suggest that the double-inducible gene activation system is efficient and highly regulated.

### REFERENCES CITED

The following references, to the extent that they provide exemplary procedural or other details supplementary to those set forth herein, are specifically incorporated herein by reference.
1. Wang Y, Xu J, Pierson T, O'Malley B W, Tsai S Y. Positive and negative regulation of gene expression in eukaryotic cells with an inducible transcriptional regulator. Gene Ther. 1997; 4:432-41.
2. Tsai S Y, O'Malley B W, DeMayo F J, Wang Y, Chua S S. A novel RU486 inducible system for the activation and repression of genes. Adv Drug Deliv Rev. 1998; 30:23-31.
3. Ngan E S, Schillinger K, DeMayo F, Tsai S Y. The mifepristone-inducible gene regulatory system in mouse models of disease and gene therapy. Semin Cell Dev Biol. 2002; 13:143-9.
4. Dong A, Hu J, Zhao L, Xu H, Liu X. Regulation and Pharmacokinetics of Inducible Recombinant TRAIL Expression. Cancer Biol Ther. 2007; 6.
5. Reboredo M, Zabala M, Mauleon I, De Las Rivas J, Kreppel F, Kochanek S, Prieto J, Hernandez-Alcoceba R, Kramer M G. Interleukin-12 inhibits liver-specific drug-inducible systems in vivo. Gene Ther. 2007.
6. Szymanski P, Kretschmer P J, Bauzon M, Jin F, Qian H S, Rubanyi G M, Harkins R N, Hermiston T W. Development and Validation of a Robust and Versatile One-plasmid Regulated Gene Expression System. Mol Ther. 2007; 15:1340-7.
7. Giannakou M E, Goss M, Jacobson J, Vinti G, Leevers S J, Partridge L. Dynamics of the action of dFOXO on adult mortality in Drosophila. Aging Cell. 2007; 6:429-38.
8. Casper K B, Jones K, McCarthy K D. Characterization of astrocyte-specific conditional knockouts. Genesis. 2007; 45:292-9.
9. Shah V R, Koster M I, Roop D R, Spencer D M, Wei L, Li Q, Schwartz R J, Chang J. Double-inducible gene activation system for caspase 3 and 9 in epidermis. Genesis. 2007; 45:194-9.
10. Ford D, Hoe N, Landis G N, Tozer K, Luu A, Bhole D, Badrinath A, Tower J. Alteration of Drosophila life span using conditional, tissue-specific expression of transgenes triggered by doxycyline or RU486/Mifepristone. Exp Gerontol. 2007; 42:483-97.
11. Kao W W. Ocular surface tissue morphogenesis in normal and disease states revealed by genetically modified mice. Cornea. 2006; 25:S7-S19.
12. Vaisanen-Tommiska M, Butzow R, Ylikorkala 0, Mikkola TS. Mifepristone-induced nitric oxide release and expression of nitric oxide synthases in the human cervix during early pregnancy. Hum Reprod. 2006; 21:2180-4.
13. Bhat R A, Stauffer B, Komm B S, Bodine P V. Regulated expression of sFRP-1 protein by the GeneSwitch system. Protein Expr Purif. 2004; 37:327-35.
14. Kanwal C, Mu S, Kern S E, Lim C S. Bidirectional on/off switch for controlled targeting of proteins to subcellular compartments. J Control Release. 2004; 98:379-93.
15. Matsumoto T, Kiguchi K, Jiang J, Carbajal S, Ruffino L, Beltran L, Wang X J, Roop DR, DiGiovanni J. Development of transgenic mice that inducibly express an active form of c-Src in the epidermis. Mol Carcinog. 2004; 40:189-200.
16. McGuire S E, Mao Z, Davis R L. Spatiotemporal gene expression targeting with the TARGET and gene-switch systems in Drosophila. Sci STKE. 2004; 2004:p16.
17. Wang L, Hernandez-Alcoceba R, Shanrlar V, Zabala M, Kochanek S, Sangro B, Kramer M G, Prieto J, Qian C. Prolonged and inducible transgene expression in the liver using gutless adenovirus: a potential therapy for liver cancer. Gastroenterology. 2004; 126:278-89.
18. Nordstrom J L. The antiprogestin-dependent GeneSwitch system for regulated gene therapy. Steroids. 2003; 68:1085-94.
19. Kyrkanides S, Miller J H, Bowers W J, Federoff H J. Transcriptional and posttranslational regulation of Cre recombinase by RU486 as the basis for an enhanced inducible expression system. Mol Ther. 2003; 8:790-5.
20. Takagi M, Yamakawa H, Watanabe T, Suga T, Junji Y. Inducible expression of long-chain acyl-CoA hydrolase gene in cell cultures. Mol Cell Biochem. 2003; 252:379-85.
21. Pollett J B, Zhu X, Gandhi S, Bali M, Masih-Khan E, Li Z, Wen X Y, Stewart A K. RU486-inducible retrovirus-mediated caspase-3 overexpression is cytotoxic to bcl-xL-expressing myeloma cells in vitro and in vivo. Mol Ther. 2003; 8:230-7.
22. Bellido C, Aguilar R, Garrido-Gracia J C, Sanchez-Criado J E. Effects of progesterone (P) and antiprogestin RU486 on LH and FSH release by incubated pituitaries from rats treated with the SERM LY11701 8-HCI and/or recombinant human FSH. J Endocrinol Invest. 2002; 25:702-8.
23. Chae J, Zimmerman L B, Grainger R M. Inducible control of tissue-specific transgene expression in Xenopus tropicalis transgenic lines. Mech Dev. 2002; 117:235-41.
24. Ngan E S, Ma Z Q, Chua S S, DeMayo F J, Tsai S Y. Inducible expression of FGF-3 in mouse mammary gland. Proc Natl Acad Sci USA. 2002; 99:11187-92.
25. Garcia E L, Mills A A. Getting around lethality with inducible Cre-mediated excision. Semin Cell Dev Biol. 2002; 13:151-8.
26. Ye X, Schillinger K, Burcin M M, Tsai S Y, O'Malley B W. Ligand-inducible transgene regulation for gene therapy. Methods Enzymol. 2002; 346:551-61.
27. Kellendonk C, Tronche F, Casanova E, Anlag K, Opherk C, Schutz G. Inducible site-specific recombination in the brain. J Mol Biol. 1999; 285:175-82.
28. Harvey D M, Caskey C T. Inducible control of gene expression: prospects for gene therapy. Curr Opin Chem Biol. 1998; 2:512-8.
29. Brocard J, Feil R, Chambon P, Metzger D. A chimeric Cre recombinase inducible by synthetic, but not by natural ligands of the glucocorticoid receptor. Nucleic Acids Res. 1998; 26:4086-90.
30. Burcin M M, O'Malley B W, Tsai S Y. A regulatory system for target gene expression. Front Biosci. 1998; 3:c1-7.
31. Clackson T. Controlling mammalian gene expression with small molecules. Curr Opin Chem Biol. 1997; 1:210-8.
32. Wang Y, DeMayo F J, Tsai S Y, O'Malley B W. Ligand-inducible and liver-specific target gene expression in transgenic mice. Nat Biotechnol. 1997; 15:239-43.
33. Wang Y, O'Malley B W, Tsai S Y. Inducible system designed for future gene therapy. Methods Mol Biol. 1997; 63:401-13.
34. Delort J P, Capecchi M R. TAXI/UAS: A molecular switch to control expression of genes in vivo. Hum Gene Ther. 1996; 7:809-20.
35. Kellendonk C, Tronche F, Monaghan A P, Angrand P O, Stewart F, Schutz G. Regulation of Cre recombinase activity by the synthetic steroid RU 486. Nucleic Acids Res. 1996; 24:1404-11.
36. Bo J, Yu W, Zhang Y M, Demayo F J, Wei L. Cardiac-specific and ligand-inducible target gene expression in transgenic mice. J Mol Cell Cardiol. 2005; 38:685-91.
37. Fan L, Freeman K W, Khan T, Pham E, Spencer D M. Improved artificial death switches based on caspases and FADD. Hum Gene Ther. 1999; 10:2273-85.
38. Shariat S F, Desai S, Song W, Khan T, Zhao J, Nguyen C, Foster B A, Greenberg N, Spencer D M, Slawin K M. Adenovirus-mediated transfer of inducible caspases: a novel “death switch” gene therapeutic approach to prostate cancer. Cancer Res. 2001; 61:2562-71.
39. Dong Z, Zeitlin B D, Song W, Sun Q, Karl E, Spencer D M, Jain H V, Jackson T, Nunez G, Nor J E. Level of endothelial cell apoptosis required for a significant decrease in microvessel density. Exp Cell Res. 2007; 313:3645-57.
40. Goggin K, Bissonnette C, Grenier C, Volkov L, Roucou X. Aggregation of cellular prion protein is initiated by proximity-induced dimerization. J Neurochem. 2007; 102:1195-205.
41. Cotugno G, Formisano P, Giacco F, Colella P, Beguinot F, Auricchio A. AP20187-mediated activation of a chimeric insulin receptor results in insulin-like actions in skeletal muscle and liver of diabetic mice. Hum Gene Ther. 2007; 18:106-17.
42. Pajvani U B, Trujillo M E, Combs T P, Iyengar P, Jelicks L, Roth K A, Kitsis R N, Scherer P E. Fat apoptosis through targeted activation of caspase 8: a new mouse model of inducible and reversible lipoatrophy. Nat Med. 2005; 11:797-803.
43. Song W, Sun Q, Dong Z, Spencer D M, Nunez G, Nor J E. Antiangiogenic gene therapy: disruption of neovascular networks mediated by inducible caspase-9 delivered with a transcriptionally targeted adenoviral vector. Gene Ther. 2005; 12:320-9.
44. Burnett S H, Kershen E J, Zhang J, Zeng L, Straley S C, Kaplan A M, Cohen D A. Conditional macrophage ablation in transgenic mice expressing a Fas-based suicide gene. J Leukoc Biol. 2004; 75:612-23.
45. Lu P D, Jousse C, Marciniak S J, Zhang Y, Novoa I, Scheuner D, Kaufman R J, Ron D, Harding H P. Cytoprotection by pre-emptive conditional phosphorylation of translation initiation factor 2. Embo J. 2004; 23:169-79.
46. Freeman K W, Gangula R D, Welm B E, Ozen M, Foster B A, Rosen J M, Ittmann M, Greenberg N M, Spencer D M. Conditional activation of fibroblast growth factor receptor (FGFR) 1, but not FGFR2, in prostate cancer cells leads to increased osteopontin induction, extracellular signal-regulated kinase activation, and in vivo proliferation. Cancer Res. 2003; 63:6237-43.
47. Vella V, Zhu J, Frasca F, Li C Y, Vigneri P, Vigneri R, Wang J Y. Exclusion of c-Abl from the nucleus restrains the p73 tumor suppression function. J Biol Chem. 2003; 278:25151-7.
48. Pownall M E, Welm B E, Freeman K W, Spencer D M, Rosen J M, Isaacs H V. An inducible system for the study of FGF signalling in early amphibian development. Dev Biol. 2003; 256:89-99.
49. Chang D W, Yang X. Activation of procaspases by FK506 binding protein-mediated oligomerization. Sci STKE. 2003; 2003:PL1.
50. Welm B E, Freeman K W, Chen M, Contreras A, Spencer D M, Rosen J M. Inducible dimerization of FGFR1: development of a mouse model to analyze progressive transformation of the mammary gland. J Cell Biol. 2002; 157:703-14.
51. Srinivasula S M, Poyet J L, Razmara M, Datta P, Zhang Z, Alnemri E S. The PYRIN-CARD protein ASC is an activating adaptor for caspase-1. J Biol Chem. 2002; 277:21119-22.
52. Li Z Y, Otto K, Richard R E, Ni S, Kirillova I, Fausto N, Blau C A, Lieber A. Dimerizer-induced proliferation of genetically modified hepatocytes. Mol Ther. 2002; 5:420-6.
53. Nor J E, Hu Y, Song W, Spencer D M, Nunez G. Ablation of microvessels in vivo upon dimerization of iCaspase-9. Gene Ther. 2002; 9:444-51.
54. Xie X, Zhao X, Liu Y, Zhang J, Matusik R J, Slawin K M, Spencer D M. Adenovirus-mediated tissue-targeted expression of a caspase-9-based artificial death switch for the treatment of prostate cancer. Cancer Res. 2001; 61:6795-804.
55. Cao T, He W, Roop D R, Wang X J. K14-GLp65 transactivator induces transgene expression in embryonic epidermis. Genesis. 2002; 32:189-90.
56. Koster M I, Kim S, Mills A A, DeMayo F J, Roop D R p63 is the molecular switch for initiation of an epithelial stratification program. Genes Dev. 2004; 18:126-31.
57. Kucera G T, Bortner D M, Rosenberg M P. Overexpression of an Agouti cDNA in the skin of transgenic mice recapitulates dominant coat color phenotypes of spontaneous mutants. Dev Biol. 1996; 173:162-73.
58. Mallet V O, Mitchell C, Guidotti J E, Jaffray P, Fabre M, Spencer D, Arnoult D, Kahn A, Gilgenkrantz H. Conditional cell ablation by tight control of caspase-3 dimerization in transgenic mice. Nat Biotechnol. 2002; 20:1234-9.

What is claimed:

1. A double-inducible gene expression system comprising:
a) a first plasmid comprising a chimeric transcription factor; and
b) a second plasmid comprising a transgene, the transgene comprising a target gene and an inducible dimerization domain, and wherein the chimeric transcription factor is capable of regulating expression of the transgene.

2. The double-inducible gene expression system of claim 1, wherein the chimeric transcription factor further comprises a Glp65 domain.

3. The double inducible gene expression system of claim 1, wherein the chimeric transcription factor further comprises a PR-LBD, and is inducible using RU486.

4. The double-inducible gene expression system of claim 1, wherein the chimeric transcription factor further comprises a K14 promoter

5. The double-inducible gene expression system of claim 1, wherein the chimeric transcription factor further comprises a Gal4 binding domain.

6. The double-inducible gene expression system of claim 1, wherein the second plasmid is induced by the chimeric transcription factor via a Gal4 binding domain.

7. The double-inducible gene expression system of claim 1, wherein the second plasmid further comprises four copies of the 17-mer Gal4 binding site in the promoter region.

8. The double-inducible gene expression system of claim 1, wherein the inducible dimerization domain is a chemical inducer of dimerization (CID) binding domain (CBD), and the domain is capable of being induced by the CID.

9. The double-inducible gene expression system of claim 1, wherein the target gene is a caspase precursor.

10. The double-inducible gene expression system of claim 9, wherein the precursor is a precursor of caspase-3 or caspase-9.

11. A double inducible gene expression system comprising:
an inducible system at a transcriptional level; and
an inducible system at a posttranslational level.

12. The double inducible gene expression system of claim 11, wherein the inducible system at the transcriptional level is an RU486-inducible system, and wherein the inducible system at the posttranslational level is a chemical inducer of dimerization (CID)-inducible system.

13. A method for generating a bigenic mouse comprising the steps of:
Delivering the double-inducible gene expression system of claim 1 to a host mouse.

14. A method for generating a bigenic mouse comprising the steps of:
a) obtaining a first transgenic mouse which expresses a first plasmid, the first plasmid comprising a chimeric transcription factor; and
b) breeding the first transgenic mouse with a second transgenic mouse which expresses a second plasmid, the second plasmid comprising a transgene, the transgene comprising a target gene and an inducible dimerization domain;
wherein the transcription factor is capable of reglating expression of the transgene.

15. The method of claim 14, wherein the chimeric transcription factor further comprises a Glp65 domain.

16. The method of claim 14, wherein the chimeric transcription factor further comprises a PR-LBD, and is inducible using RU486.

17. The method of claim 14, wherein the chimeric transcription factor further comprises a K14 promoter

18. The method of claim 14, wherein the chimeric transcription factor further comprises a Gal4 binding domain.

19. The method of claim 14, wherein the second plasmid is induced by the chimeric transcription factor via a Gal4 binding domain.

20. The method of claim 14, wherein the second plasmid further comprises four copies of the 17-mer Gal4 binding site in the promoter region.

21. The method of claim 14, wherein the inducible dimerization domain is a chemical inducer of dimerization (CTD) binding domain (CBD), and the domain is capable of being induced by CID.

22. The method of claim 14, wherein the target gene is a caspase precursor.

23. The method of claim 14, wherein the caspase is a precursor of caspase-3 or caspase-9.
